(19) United States
(12) Patent Application Publication
(10) Pub. No.: US 2023/0089090 A1
(43) Pub. Date: Mar. 23, 2023

(54) Composition for Treating Viral Infections

(71) Applicant: Peter HOFLEITNER, Annapolis, MD (US)

(72) Inventor: Peter HOFLEITNER, Annapolis, MD (US)

(21) Appl. No.: 17/922,676

(22) PCT Filed: May 25, 2021
(86) PCT No.: PCT/US2021/034002

§ 371 Date: Nov. 1, 2022

Related U.S. Application Data
(60) Provisional application No. 63/041,471, filed on Jun. 19, 2020.

(51) Int. Cl. A61K 31/375; A61K 31/352; A61P 31/14

(52) U.S. Cl. CPC A61K 31/375; A61K 31/352; A61P 31/14

(57) ABSTRACT

A composition of a therapeutically effective amount of luteolin, quercetin, kaempferol, and vitamin C, and a therapeutically acceptable carrier for preventing or treating viral infections. A method of preventing or treating a viral infection in a subject by administering the composition to a subject. The viral infection may be a coronavirus infection.

The invention relates to anti-viral compositions and methods for treating viral infections.

Coronaviruses are a family of viruses that may cause disease in animals or humans. Several coronaviruses are known to cause respiratory infections in humans ranging from the common cold to more severe diseases such as Middle East Respiratory Syndrome (MERS) and Severe Acute Respiratory Syndrome (SARS).

The world is currently experiencing a coronavirus pandemic due to a novel coronavirus whose official WHO name is coronavirus disease (or “COVID-19”). The coronavirus itself has official WHO name SARS-CoV-2. Since the emergence of SARS-CoV-2 the world is feverishly developing cures and vaccines to mitigate the pandemic. FDA approved pharmaceuticals and vaccines take time to develop and once approved may not be available broadly at affordable cost.

Thus, a need exists for naturally occurring substances that reduce or inhibit the pathogenicity of SARS-CoV-2, in particular substances that can be offered as orally administered supplements and made available quickly.

### SUMMARY

The invention is an anti-viral composition containing effective amounts of kaempferol, vitamin C (ascorbic acid), luteolin, and quercetin, and a pharmaceutically acceptable carrier. In one aspect, the composition includes 80 mg to 220 mg kaempferol, 475 mg to 525 mg vitamin C, 80 mg to 220 mg luteolin, and 90 mg to 310 mg quercetin or corresponding relative amounts of these four components.

In another aspect, the composition includes luteolin, quercetin, kaempferol, vitamin C are present in a mass ratio of from about 2:1:2:5 to about 1:3:1:5. In one aspect, the composition includes 180 mg to 220 mg kaempferol, 475 mg to 525 mg vitamin C, 180 mg to 220 mg luteolin, and 90 mg to 110 mg quercetin, preferably 190 mg to 210 mg kaempferol, 490 mg to 510 mg vitamin C, 190 mg to 210 mg luteolin, and 95 mg to 105 mg quercetin. In another aspect, the composition contains about 200 mg kaempferol, about 500 mg vitamin C, about 200 mg luteolin, and about 100 mg quercetin. In yet another aspect, the luteolin, quercetin, kaempferol, vitamin C are present in a mass ratio of about 2:1:2:5.

In another aspect, the composition contains 80 mg to 120 mg kaempferol, 475 mg to 525 mg vitamin C, 80 mg to 120 mg luteolin, and 290 mg to 310 mg quercetin. In yet another aspect, the composition contains about 100 mg kaempferol, about 500 mg vitamin C, about 100 mg luteolin, and about 300 mg quercetin. In yet another aspect, the luteolin, quercetin, kaempferol, vitamin C are present in a mass ratio of about 1:3:1:5.

In an advantageous aspect, the formulation enhances bioavailability (absorption and retention) of luteolin and quercetin.

In yet another aspect, the composition is formulated for oral administration, preferably as a tablet or capsule.

In another aspect, the invention is a method of preventing or treating a viral infection in a subject by administering an effective amount of the composition as described above. In another aspect, the viral infection is a coronavirus infection. In yet another aspect, the viral infection is COVID-19, HIV, human and avian influenza, herpes, Epstein-Barr, leukemia, dengue, Ebola, hepatitis B, measles, West Nile, Zika, RSV, SARS, MERS, or Marburg virus. In yet another aspect, the viral infection is COVID-19 due to the coronavirus SARS-CoV-2. In yet another aspect, the viral infection is an influenza infection.

In an aspect of the method, the composition is administered once every 3 to 4 hours, alternatively twice a day or once every 8-12 hours. In another aspect, the composition is administered at an elevated dosage within 1 to 2 days after onset of the viral infection, preferably within 1-4 hours or immediately at onset of viral infection symptoms.

In another aspect of the method, the subject has a preexisting condition or comorbidity, e.g., hypertension, diabetes, coronary heart disease, obesity, cerebrovascular illness, tobacco smoking, chronic obstructive pulmonary disease (COPD), or kidney dysfunction.

### BRIEF DESCRIPTION OF THE FIGURES

FIG. 1 is an image of a subject with a herpes simplex blister that remained very small due to proactive treatment.

FIG. 2 is an image of a subject with a herpes simplex blister that was untreated for 48 hours.

FIG. 3 is an image of a subject with a herpes simplex blister that had rapidly grown in size prior to treatment.

### DETAILED DESCRIPTION

Those skilled in the art will understand that this invention may be embodied in many different forms and should not be construed as limited to the embodiments set forth in this application. Rather, these embodiments are provided so that this disclosure will fully convey the invention to those skilled in the art. Many modifications and other embodiments of the invention will come to mind in one skilled in the art to which this invention pertains having the benefit of the teachings presented herein.

Luteolin (2-(3,4-dihydroxyphenyl)-5,7-dihydroxy-4-chromenone) is a natural flavonoid found in a number of plant sources, including celery, broccoli, green pepper, parsley, thyme, dandelion, perilla, chamomile tea, carrots, olive oil, peppermint, rosemary, navel oranges, and oregano.

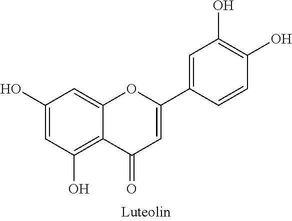 [SMILES: O=c1cc(-c2ccc(O)c(O)c2)oc2cc(O)cc(O)c12]

Quercetin (2-(3,4-dihydroxyphenyl)-3,5,7-trihydroxy-4H-chromen-4-one) is a natural flavonoid found in a variety of plants such as red onions, kale, sorrel, radish leaves, dill, and cilantro. Preferably, the quercetin is in the form of a hydrate, preferably quercetin dihydrate.

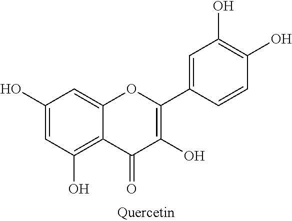 [SMILES: O=c1c(O)c(-c2ccc(O)c(O)c2)oc2cc(O)cc(O)c12]

Kaempferol (3,4′,5,7-tetrahydroxyflavone) is a natural flavonoid found in a variety of plants such as kale, beans, tea, spinach, and broccoli.

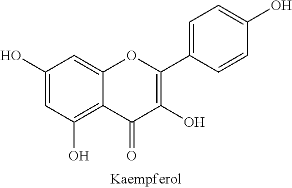 [SMILES: O=c1c(O)c(-c2ccc(O)cc2)oc2cc(O)cc(O)c12]

Vitamin C ((R)-3,4-dihydroxy-5-((S)-1,2-dihydroxyethyl)furan-2(5H)-one) refers to the L-enantiomer of ascorbic acid and its oxidized forms, such as dehydroascorbate (DHA), and pharmaceutically acceptable salts thereof, e.g., sodium or calcium salts.

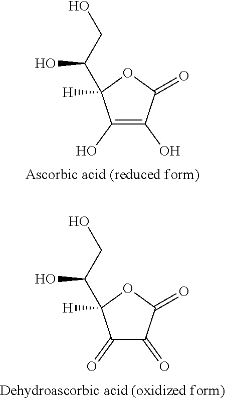 [SMILES: O=C1O[C@H]([C@@H](O)CO)C(O)=C1O ; O=C1O[C@H]([C@@H](O)CO)C(=O)C1=O]

Luteolin is a furin inhibitor, and quercetin targets ACE2 expression. Biochemical interrogation of human furin shows that luteolin inhibits furin's enzyme activity in an uncompetitive manner. If furin is inhibited it cannot effectively cleave proteins.

Furin is an invasion-promoting protease for SARS-COV-2. It preactivates the S protein of SARS-COV-2 by cleaving it along its furin motif. This allows the S protein to effectively bind to its receptor human ACE2 (hACE2), enabling efficient cell entry. If the S protein is not cleaved with furin then cell entry is less effective and, according to research, may results in a 100-fold reduced infection pathway.

Quercetin targets the ACE2 expression and can further reduce the pathogenicity of SARS-COV-2. According to present research results, Quercetin exhibits better potential inhibition than hydroxy-chloroquine against COVID-19 main protease active site and ACE2.

In combination luteolin and quercetin are potent flavonoids that can reduce pathogenicity of the SARS-COV-2 virus by targeting preactivation of the S protein and ACE2 receptor binding of the host cell. The present invention advantageously enhances effectiveness of both flavonoid's absorption and retention in vivo.

The efficient invasion of host cells by the SARS-CoV-2 is further enhanced by the presence of the unexpected furin cleavage site, which is cleaved during the biosynthesis (Glinsky, Biomedicines 2020, 8(5), 129; Walls et al., Cell 2020, 181, 281-292). This novel feature distinguishes the previously known SARS-CoV and the newly emerged SARS-CoV-2 viruses and possibly contributes to the expansion of the cellular tropism of the SARS-CoV-2 (Walls et al., 2020). Collectively, these observations identified protein products of the human genes ACE2 and FURIN as the high-affinity receptor (ACE2) and invasion-promoting protease (FURIN) acting as the principal mediators of the SARS-CoV-2 invasion into human cells. See arxiv.org/pdf/2003.13665.pdf.

The “furin-like cleavage site” recently discovered in SARS-CoV-2 spike proteins may explain the viral life cycle and pathogenicity of the virus, say researchers. “[The furin activation site] sets the virus up very differently to SARS, in terms of its entry into cells, and possibly affects virus stability and hence transmission.” See www.medicalnewstoday.com/articles/why-does-sars-cov-2-spread-so-easily#Spike-protein-on-the-new-coronavirus.

The entry of SARS-CoV-2 into cells is mediated by the efficient binding of the spike (S) viral protein, a 1273 amino acid long protein which belongs to the viral envelope and protrudes outwards with a ‘corona’ like appearance, to the angiotensin converting enzyme 2 (ACE2) receptors. See www.ncbi.nlm.nih.gov/pmc/articles/PMC7167588/#bib0044.

Unlike SARS-CoV, cell entry of SARS-CoV-2 is preactivated by proprotein convertase furin, reducing its dependence on target cell proteases for entry. The high hACE2 binding affinity of the RBD, furin preactivation of the spike, and hidden RBD in the spike potentially allow SARS-CoV-2 to maintain efficient cell entry while evading immune surveillance. See www.pnas.org/content/117/21/11727

Some of the most pathogenic forms of influenza have similar cleavage sites, which can be acted upon by furin and other cellular proteases. The ubiquitous expression of cellular proteases across cell types increases the potential for the virus to successfully infiltrate the host. See www.assaygenie.com/how-furin-and-ace2-interact-with-the-spike-on-sarscov2.

Intriguingly, accumulating evidence suggests that inhibition of the virus dependency factor furin represents another efficient and broadly active mechanism of antiviral immunity.

Notably, furin expression is induced by hypoxia, as all three FUR promoters harbour binding sites for the hypoxia-inducible factor-1 (HIF-1).31 Thus, furin-mediated vascularisation may preferentially occur in otherwise growth-restricted hypoxic tumors. Interestingly, hypoxia also results in subcellular relocalisation of furin to the cell surface, which may further enhance processing of growth factors and other extracellular tumorigenic precursor proteins. The ability of viruses to exploit furin may have drastic effects on their pathogenicity. See www.ncbi.nlm.nih.gov/pmc/articles/PMC6682551/.

The broad role of furin in activating bacterial toxins is exceeded by its role in activating numerous pathogenic viruses. Many pathogenic viruses, including avian influenza virus, HIV-1, measles virus and RSV, express envelope glycoproteins that must be cleaved at consensus furin sites to form the mature and fusogenic envelope glycoprotein. See www.ncbi.nlm.nih.gov/pmc/articles/PMC1964754/.

The ubiquitous presence of furin and related PCs throughout the cells of the body makes these proteases vulnerable to being exploited by viruses. See www.ncbi.nlm.nih.gov/pmc/articles/PMC6784293/

Patients with hypertension, diabetes, coronary heart disease, cerebrovascular illness, COPD, and kidney dysfunction have worse clinical outcomes when infected with SARS-CoV-2, for unknown reasons. The purpose of this review is to summarize the evidence for the existence of elevated plasmin(ogen) in COVID-19 patients with these comorbid conditions. Plasmin, and other proteases, may cleave a newly inserted furin site in the S protein of SARS-CoV-2, extracellularly, which increases its infectivity and virulence. Hyper-fibrinolysis associated with plasmin leads to elevated D-dimer in severe patients. The plasmin(ogen) system may prove a promising therapeutic target for combating COVID-19. The envelope proteins of numerous viruses, such as HIV, human and avian influenza, herpes, Epstein-Barr, leukemia, dengue, Ebola, hepatitis B, measles, West Nile, Zika, RSV, SARS, MERS, and Marburg virus, are cleaved by intracellular furin-like proteases. Furin is predominately expressed in human alveolar type II (AT2) cells in the respiratory system. The presence of a polybasic cleavage site that can be cleaved by furin-like proteases, is a signature of several highly pathogenic avian influenza viruses (82). Similarly, the S protein of SARS-CoV-2 harbors a furin cleavage site at the S1/S2 boundary. The almost ubiquitous and diverse expression of furin-like proteases could lead to increasing SARS-CoV-2 cell and tissue tropism and transmissibility, and enhance its pathogenicity. See journals.physiology.org/doi/pdf/10.1152/physrev.00013.2020.

Coronaviruses have been reported to enter host cells in two distinct routes. The S1 subunit of SARS-CoV-2 may bind ACE2 receptor in lung, renal and cardiac tissue, liver, testis and intestinal epithelia to mediate the virus fusion and lead to the related symptoms. Alternatively, the S protein could be cleaved by S-activating protease co-expressed with the host cell receptor, thereby inducing the direct fusion of viral and cellular membrane furin is a key factor for viral entry of human immunodeficiency virus type-1 (HIV-1) through cleaving its envelope glycoprotein. In addition, furin could proteolytically activate membrane fusion activity of influenza viruses through cutting their hemagglutinin [this] expression of endopeptidases may provide a 100-fold higher efficient infection pathway than receptor-mediated endocytosis does. Whether the treatment of furin inhibitors could benefit these SARS-CoV-2-infected patients deserves further researches. See journals.physiology.org/doi/pdf/10.1152/physrev.00013.2020.

Observations suggest that inhibitors of furin-like enzymes may contribute to inhibiting virus propagation. See www.sciencedirect.com/science/article/pii/S0166354220300528?via %3Dihub. Luteolin restricts dengue virus replication through inhibition of the proprotein convertase furin. See dr.ntu.edu.sg/bitstream/10220/44137/1/Luteolin %20restricts %20dengue %20virus %20rep lication %20through %20inhibition %20 of %20the %20proprotein %20convertase %20furin.pdf.

During the virus life cycle, the host protease furin cleaves the pr moiety from prM protein of immature virus particles in the trans-Golgi network to produce mature virions. Analysis of virus particles from luteolin-treated cells revealed that prM was not cleaved efficiently. Biochemical interrogation of human furin showed that luteolin inhibited the enzyme activity in an uncompetitive manner, with Ki value of 58.6 μM, suggesting that treatment may restrict the virion maturation process. The envelope glycoprotein of human immunodeficiency virus (HIV) initiates infection by mediating fusion of the viral envelope with the cell membrane. Fusion activity requires proteolytic cleavage of the gp160 protein into gp120 and gp41 at a site containing several arginine and lysine residues. The glycoprotein of HIV-1, which has the same protease recognition motif as the FPV haemagglutinin, is also activated by furin. See www.ncbi.nlm.nih.gov/pubmed/28389141.

Luteolin treatment of HIV-1 infected lymphocytes also showed inhibition in cell aggregation/syncytia similar to that produced by DRB and cell control, suggesting that viral envelope (gp120) protein expression on cell surfaces is impaired. . . . the glycoprotein of HIV-1 . . . is also activated by furin. See www.ncbi.nlm.nih.gov/pmc/articles/PMC3227592/; pubmed.ncbi.nlm.nih.gov/1360148/.

According to in silico results, quercetin have a better affinity against COVID-19 protease than hydroxy-chloroquine. The obtained results show also that Quercetin exhibited as the best potential inhibitors against protease of COVID-19. See www.researchgate.net/publication/340911244_In-Silico_Identification_of_Potent_Inhibitors_of_COVID-19_Main_Protease_Mpro_and_Angiotensin_Converting_Enzyme_2_ACE2_from_Natura 1_Products_Quercetin_Hispidulin_and_Cirsimaritin_Exhibited_Better_Poten.

Quercetin appears to inhibit expression of several potential coronavirus infection-promoting genes. Present analyses identify vitamin D and quercetin as promising pandemic mitigation agents. Quercetin seems to target ACE2 expression. See arxiv.org/pdf/2003.13665.pdf

Quercetin has unique biological properties that may improve mental/physical performance and reduce infection risk. These properties form the basis for potential benefits to overall health and disease resistance, including anti-carcinogenic, anti-inflammatory, antiviral, antioxidant, and psychostimulant activities, as well as the ability to inhibit lipid peroxidation, platelet aggregation and capillary permeability, and to stimulate mitochondrial biogenesis. See www.ncbi.nlm.nih.gov/pmc/articles/PMC4808895/

Improving bio-availability during oral administration is important. The simultaneous ingestion of quercetin with vitamin C, folate and additional flavonoids improves bioavailability. See www.ncbi.nlm.nih.gov/pmc/articles/PMC4808895/.

Quercetin metabolites appeared in plasma after 30 min of ingestion, but a significant amount was excreted over a 24-h period. This indicates rapid clearance and a short half-life of quercetin in the blood. See www.ncbi.nlm.nih.gov/pmc/articles/PMC6835347/.

Luteolin accumulated in cells after 24-h incubation, whereas quercetin disappeared completely from cell fractions and culture medium. Addition of ascorbic acid prevented the disappearance of quercetin and allowed it to exert its cytotoxicity (similar to luteolin) at >10 μM. It was apparent that ascorbic acid protected quercetin from auto-oxidation to allow its accumulation in cell fractions and the culture medium upon 24-h incubation. O-Methylated metabolites of quercetin also accumulated at higher levels as compared with those in the absence of ascorbic acid. See www.ncbi.nlm.nih.gov/pmc/articles/PMC5598286/.

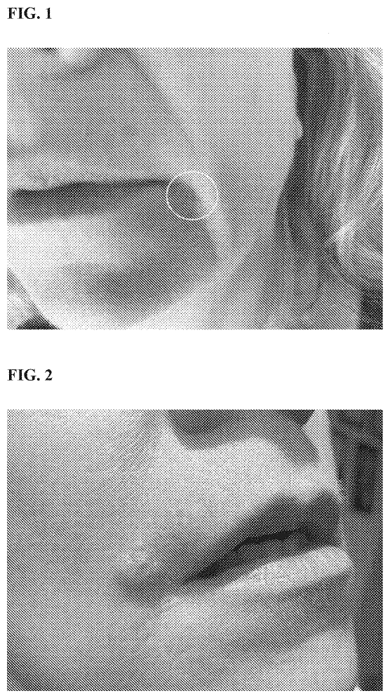

A defined composition consisting of luteolin, quercetin, and kaempferol at the molar ratio of 1:1:2. In vitro cytotoxicity assays found that, as single compounds, luteolin, quercetin, and kaempferol have weak to modest activities in PCa cells. For example, the half minimal inhibitory concentration (IC50) of luteolin, quercetin, and kaempferol in androgen receptor (AR)-positive C4-2 cells is 114.02, 55.25, and 157.81 μM, respectively. In comparison, ProFine exhibited enhanced cytotoxicity compared to any of the three individual components, with the IC50 of 16.56 μg/ml in C4-2 cells (equivalent to 14.28, 14.28, and 28.60 μM of luteolin, quercetin, and kaempferol, respectively). Indeed, isobologram analysis showed that the combination index (CI) achieved as low as 0.11 when the three ingredients were used at low concentrations, indicating a strong synergy among them (see FIG. 1B; Table S1 in www.sciencedirect.com/science/article/pii/S1476558618302653).

Administration of both flavonols simultaneously revealed that kaempferol blocked the efflux of quercetin, allowing quercetin to remain inside and exert its effects (Guohua, Gallegos, & Morris, 2011). See www.ncbi.nlm.nih.gov/pmc/articles/PMC3601579/.

Oral ProFine (a standardized composition of luteolin, quercetin, and kaempferol) was shown to be safe in rodent models when administered at high doses up to 400 mg/kg. See www.sciencedirect.com/science/article/pii/S1476558618302653.

Quercetin doses ranged from 250 to 5000 mg/day (Table 2). There were three patients per dose group, with the exception of three dose levels (2000, 3000, and 4000 mg/day), which had only two patients per group because of withdrawal from the study. High doses were well tolerated, with no adverse events or signs of toxicity. See www.ncbi.nlm.nih.gov/pmc/articles/PMC5590840/.

Kaempferol was safely administered in daily oral doses of 50, 100, and 200 mg/kg body weight. See www.sciencedirect.com/science/article/abs/pii/S0014299910012069

Major adverse effects had not been observed in children who took 10 mg/kg of luteolin (ClinicalTrials.gov identifier: NCT01847521) (Taliou et al., 2013). Although it is not a cardiovascular clinical research, 10 mg/kg may be used as a reference dosage of safety in future CVD trials. See www.ncbi.nlm.nih.gov/pmc/articles/PMC5635727/.

A “pharmaceutically acceptable carrier” includes any additive, adjuvant, diluent, or excipient used for pharmaceutical or nutraceutical dosage forms.

Formulations for oral administration include capsules, tablets, pills, powders, and granules. Such solid dosage forms might include a pharmaceutically acceptable excipient or carrier, e.g., fillers or extenders such as starches, lactose, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents such as cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof. Capsules, tablets, and pills may also contain buffering agents.

Soft and hard-filled gelatin capsules may include excipients such as lactose or milk sugar as well as high molecular weight polyethylene glycols. Tablets, dragees, capsules, pills, and granules can be prepared with coatings and shells such as enteric coatings and other coatings well known in the pharmaceutical formulating art.

In one aspect, the pharmaceutically acceptable composition may be administered with or without food.

The term “treating” or “treatment” means reversing, alleviating, or inhibiting the progress of a disease or disorder, or one or more symptoms thereof. In some aspects, treatment may be administered after one or more symptoms have developed. In other aspects, treatment may be administered in the absence of symptoms. For example, treatment may be administered to a susceptible individual prior to the onset of symptoms (e.g., due to a history of symptoms and/or due to genetic or other susceptibility factors). Treatment may also be continued after symptoms have resolved, for example to prevent or delay their recurrence.

“Preventing” or “prevention” refers to use as a prophylactic for reducing the risk of acquiring a disease or disorder, i.e., causing at least one of the clinical symptoms of the disease not to develop in a patient that may be exposed to or predisposed to the disease but does not yet experience or display symptoms of the disease.

The subject of treatment is a mammal, preferably a human subject.

The following examples serve to illustrate certain aspects of the disclosure and should not be construed as limiting the claims. The contents of all references, pending patent applications and published patents, cited throughout this application are hereby expressly incorporated by reference.

### Examples

### Example 1: COVID-19

Two subjects presenting symptoms of COVID-19 (dry cough and shortness of breath) are administered a mixture of 200 mg kaempferol, 500 mg vitamin C, 200 mg luteolin, and 100 mg quercetin within 4 hours of the first presentation of symptoms.

TABLE 1

| Ingredient | Units | Amounts |
|---|---|---|
| Kaempferol | 2 | 200 mg |
| Vitamin C | 5 | 500 mg |
| Luteolin | 2 | 200 mg |
| Quercetin | 1 | 100 mg |

Both subjects ingested the mixture every 4 hours for a total of 4 daytime doses. In both subjects, the symptoms subsided after 1 day and disappeared entirely after 3 days.

### Example 2: COVID-19

A 32-year old subject tested positive for COVID-19. Approximate date of symptoms on-set: 1/27/21. Light symptoms, sore throat and runny nose on Sunday 1/31/21. Date of diagnosis was 2/1/21. The subject began treatment on 2/3/2021 taking 6 pills a day (two pills 3 times a day). The pills were formulated to include the components as shown in Table 2 with excipients in a gel capsule.

TABLE 2

| Ingredient | Units | Amounts |
|---|---|---|
| Vitamin C (ascorbic acid) | 5 | 500 mg |
| Quercetin dihydrate | 3 | 300 mg |
| Kaempferol 98% | 1 | 103 mg |
| Luteolin | 1 | 100 mg |

No major immediate improvements but symptoms did not worsen, were gone after a couple days, and did not return thereafter. The subject continued the treatment and finished an entire bottle per recommendation even after no longer having symptoms of COVID-19. The subject suffered no lingering effects from COVID-19 after over 3 months had passed after completing treatment.

### Example 3: COVID-19 (Control)

A 29-year old subject experienced symptoms of COVID-19, with approximate date of symptoms onset: 11/27/20. Date of diagnosis was 11/30/20. The subject experienced light symptoms that worsened over a period of a few days. The subject developed fever and cough. The subject experienced slow recovery. Approximate date recovered: 12/9/20.

### Example 4: Influenza-Like Symptoms

A 59-year old male subject was susceptible to the flu or flu-like symptoms. In the past the subject experienced flu-like symptoms of varying degree and duration two to three times per year. Each occurrence was typically 2 to 4 weeks long. Symptoms included sore throat, ear pain, stuffy nose, swollen glands, congested chest. Typical progression was initial slight onset of ear pain, followed by swollen glands and sore throat with increasing ear pain. The symptoms typically progressed to include thick chest congestion with hard mucus/phlegm in the upper and lower breathing tubes and stuffy nose.

The subject began treatment with capsules according to Table 2 above upon onset of slight ear pain, and continued treatment for a week after symptoms disappeared. Once this pattern of early treatment began, the subject no longer experienced the flu-like symptoms that had been common in past flu seasons.

### Example 4: Herpes Simplex

A 58-year old female subject was susceptible to herpes simplex virus type 1 (HSV-1), frequently developing blisters around the lips that can cluster and grow very large. If left untreated it would normally take 4 to 5 weeks before the blisters disappear.

Two tests were conducted on the female subject. In the first test the female subject immediately started treatment with capsules according to Table 2 (two capsules 3 times a day) as soon as a blister was detected. With this proactive treatment the blister remained very small (see FIG. 1), dried up within 24 hours, and disappeared within a week.

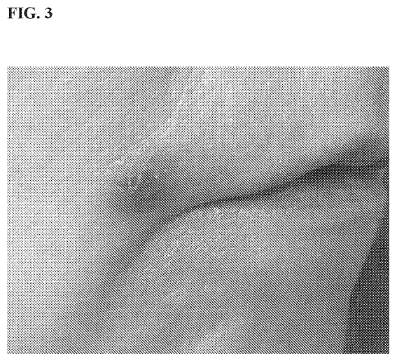

In the second test the female subject left a blister (see FIG. 2) untreated for 48 hours. The untreated blister rapidly grew in size (see FIG. 3). After 48 hours the female subject started treatment with the capsules according to Table 2 (two capsules 3 times a day). Two 2 days after the treatment (4 days after initial onset) the blister had dried up. Blisters of this size on the female subject typically disappear completely within 8 days of treatment start.

1. A composition comprising a therapeutically effective amount of luteolin, quercetin, kaempferol, vitamin C, and a pharmaceutically acceptable carrier.

2. A composition according to claim 1, comprising 80 mg to 220 mg kaempferol, 475 mg to 525 mg vitamin C, 80 mg to 220 mg luteolin, and 90 mg to 310 mg quercetin.

3. A composition according to claim 1, wherein the luteolin, quercetin, kaempferol, vitamin C are present in a mass ratio of from about 2:1:2:5 to about 1:3:1:5.

4. A composition according to claim 2, comprising 180 mg to 220 mg kaempferol, 475 mg to 525 mg vitamin C, 180 mg to 220 mg luteolin, and 90 mg to 110 mg quercetin.

5. A composition according to claim 4, comprising about 200 mg kaempferol, about 500 mg vitamin C, about 200 mg luteolin, and about 100 mg quercetin.

6. A composition according to claim 3, wherein the luteolin, quercetin, kaempferol, vitamin C are present in a mass ratio of about 2:1:2:5.

7. A composition according to claim 2, comprising 80 mg to 120 mg kaempferol, 475 mg to 525 mg vitamin C, 80 mg to 120 mg luteolin, and 290 mg to 310 mg quercetin.

8. A composition according to claim 7, comprising about 100 mg kaempferol, about 500 mg vitamin C, about 100 mg luteolin, and about 300 mg quercetin.

9. A composition according to claim 3, wherein the luteolin, quercetin, kaempferol, vitamin C are present in a mass ratio of about 1:3:1:5.

10. A composition according to claim 1, wherein the composition is formulated for oral administration.

11. A composition according to claim 1, wherein the composition is a tablet or capsule.

12. A method of preventing or treating a viral infection in a subject, comprising administering an effective amount of a composition according to claim 1 to a subject in need thereof.

13. A method according to claim 12, wherein the viral infection is due to a coronavirus, HIV, human and avian influenza, herpes, Epstein-Barr, leukemia, dengue, Ebola, hepatitis B, measles, West Nile, Zika, RSV, SARS, MERS, or Marburg virus.

14. A method according to claim 13, wherein the viral infection is a coronavirus infection and the coronavirus is SARS-CoV-2.

15. A method according to claim 13, wherein the viral infection is a herpes infection.

16. A method according to claim 13, wherein the viral infection is an influenza infection.

17. A method according to claim 12, wherein the composition is administered once every 3 to 4 hours.

18. A method according to claim 12, wherein the composition is administered within 1 to 2 days after onset of the viral infection.

19. A method according to claim 12, wherein the composition is administered at an elevated dosage at onset of viral infection symptoms.

20. A method according to claim 12, wherein the subject has hypertension, diabetes, coronary heart disease, obesity, cerebrovascular illness, tobacco smoking, chronic obstructive pulmonary disease (COPD), or kidney dysfunction.
